(19) United States
(12) Patent Application Publication
(10) Pub. No.: US 2026/0111598 A1
(43) Pub. Date: Apr. 23, 2026

(54) SYSTEM AND METHOD FOR CENTRAL PATIENT ACCESS TO A DISTRIBUTED ELECTRONIC HEALTH RECORD SYSTEM NETWORK

(71) Applicant: EPIC SYSTEMS CORPORATION, Verona, WI (US)

(72) Inventors: David Fuhrmann, Verona, WI (US); Trevor Berceau, Verona, WI (US); Haruko Okamoto, Verona, WI (US); Zachary Walters, Verona, WI (US); Benjamin Heiligenthal, Verona, WI (US); William Subach, Verona, WI (US)

(21) Appl. No.: 19/363,561

(22) Filed: Oct. 20, 2025

Related U.S. Application Data
(60) Provisional application No. 63/711,017, filed on Oct. 23, 2024.

(51) Int. Cl. G06F 21/62; G06F 16/27; G16H 10/60

(52) U.S. Cl. CPC G06F 21/6245; G06F 16/27; G16H 10/60

(57) ABSTRACT

A distributed network of electronic health record systems comprises a plurality of electronic health record systems connected in a network, each of the electronic health record systems adapted to communicate with other electronic health record systems in the network via secure communication links; and a central network node in communication with each of the plurality of electronic health record systems. The central network node is configured to display a user interface to a patient, the user interface configured to receive data from the patient to authenticate an identity of the patient; and to establish a central patient account for the patient, the central patient account being pre-authorized to access each of the plurality of electronic health record systems, and to change patient data in an electronic health record associated with the authenticated patient requesting the change.

### CROSS-REFERENCE TO RELATED APPLICATIONS

This application claims the benefit of U.S. Provisional Patent Application Ser. No. 63/711,017, filed on Oct. 23, 2024, and entitled “SYSTEM AND METHOD FOR CENTRAL PATIENT ACCESS TO A DISTRIBUTED ELECTRONIC HEALTH RECORD SYSTEM NETWORK,”which is herein incorporated by reference in its entirety.

### FIELD

The disclosure addresses systems and methods for verifying patient identity, authenticating patients, and synchronizing patient data among databases in a distributed network of healthcare information systems operated by different healthcare organizations or entities.

### BACKGROUND

Electronic health records (EHRs) have been used to store patient data since at least the 1960s, and over time have become the standard method for maintaining medical records. Currently, healthcare organizations typically maintain EHRs for their patients in healthcare information systems, which maintain a database of patient data specific for the patients of that organization, and which are often connected in a distributed network with other healthcare organizations using the same healthcare information system software and/or sharing information across the network with other EHR systems via shared standards. When a patient is seen by more than one organization, their records can therefore be maintained in databases for each organization. Despite the existing connectivity between the healthcare organizations, and the importance of portability of healthcare records, there are a number of impediments to easily accessing, maintaining, and sharing the patient data in EHRs, even between healthcare organizations that use the same healthcare information system software, and have the ability to communicate via a network or the internet.

For example, because patient consent may be required to share patient data, it is not possible to simply synchronize data among networked or connected healthcare information systems. Rather, when a patient authorizes access to their patient records, the healthcare information system at the point of care can be authorized to “pull” data corresponding to the authorizing patient from other healthcare organizations. If other connected healthcare information systems have not received consent from the patient, new data that is acquired at the point of care may not be shared back with other connected healthcare information systems. Therefore, data stored at one organization in the network may vary from data stored at another organization in the network.

Because of the need for authorization by the patient to “pull” treatment data from other organizations, these processes, moreover, typically are restricted to time frames when the patient is being treated by a healthcare organization. Healthcare providers, therefore, only access, or change, patient treatment data, and other data like the patient's name, address, phone number, or insurance information, if the patient is currently under treatment. The patient's data, therefore may be out of date when the patient is seen at another organization, and the previous treating organization may therefore not have access to important updates to the patient's clinical or personal data unless it successfully retrieves it the next time they see the patient for treatment.

Additionally, when sharing of data is allowed, data is typically accessed by querying the systems operated by other healthcare organizations through network or internet connections. Running these queries too frequently or across too many systems on the network creates high levels of unnecessary traffic, decreasing speed and causing other network inefficiencies. Conversely, relevant data updates can be missed when queries are not frequent enough, and important data can therefore be missing from the patient record.

Another problem with existing systems is that when a patient is seen at a new healthcare organization that does not have established links to the patient's record, queries for patient data are often filtered or focused by algorithms that identify places where the patient is likely to have been seen in the past, such as organizations near their home or work address, or local Health Information Exchanges. The system can then query all identified relevant healthcare organizations corresponding to the patient's electronic medical record. These approaches can miss data in situations where, for example, a patient changes their name or moves, which can make it difficult to identify and retrieve aspects of the patient's historical medical information. Additionally, expanding the search to query all organizations on the network is not practical, because searches of this type introduce a high volume of unnecessary traffic onto the interoperability network, negatively impacting performance.

Further, differences in how a patient is registered at different organizations on the network can be problematic. For example, the patient's name may be slightly different due to a spelling error, or very different after, for example, a name change or marriage. When patient identifying information is incorrect, it can hinder the process of identifying records for the patient in a query.

Another problem with pre-existing networked systems is that, although the data is updated by the healthcare organization querying to update the patient data, the querying system will “pull” data from other organizations and may not have authorization to “push” or provide updated information to the system that is being queried and have that system update the chart. If, for example, the querying organization knows that the patient has had a name change, that organization cannot change the data in the patient's record at any other organization, even if it pushes the update back to other connected healthcare organizations. The data that is maintained in other installations in the network, therefore, frequently is not updated during these processes, which can lead to mismatched patient data across organizations. These mismatches can therefore make it difficult to match patient records, and therefore hinders efforts to synchronize data.

Another problem with keeping patient data up to date at healthcare organizations is that healthcare organizations often have unique standards, requirements or restrictions associated with accepting external or patient-entered data. In some cases, these restrictions are critical for operational reasons. For example, updating a patient address without verification may, in some cases, invalidate insurance from some payers. In other cases, the restrictions may be related to clinical reasons. For example, when removing a blood pressure medication from a patient's medication list entirely, the healthcare organization may require that medical personnel see the patient to discuss whether the medication change is appropriate. Therefore the patient in some cases may not be able to provide the most recent data, and in others they may be able to submit the most recent data but the data may not be immediately visible to the patient or staff at healthcare organizations.

Additionally, individual healthcare organizations can store individual data elements using different internal identifiers. For example, different organizations may use different names or codes for identifying medical conditions or treatments. It can, therefore, be difficult to translate the data held at one healthcare organization into useful information for storage in an EHR held at another healthcare organization.

Identifying that an account holder is who they assert that they are is also problematic in sharing healthcare data. Traditional approaches of secure identity verification often require an in-person interaction to present official identifying documents, or a multi-step digital process involving steps like image analysis from multiple angles or a series of obscure questions related to the person's history, such as financial information, past addresses, past phone numbers, automobile registration and licensing, and other information. The difficulty associated with answering these questions often causes patients trying to register to stop partway through and not obtain a verified identity, which can therefore prevent access to online records.

Electronic access to electronic healthcare systems by patients, moreover, is typically specific to each healthcare organization. That is, each healthcare organization enables a patient to establish an account for accessing their own health records through a user interface at a website or other networked link. The patient, therefore, typically has a separate account associated with each healthcare organization. Each of these organizations require the patient to establish a username and password. Upon entry to the system, the patient can access and change the information associated with their activities with the electronic health record system operated by the healthcare organization providing access, but does not typically have access to make changes to data held by other healthcare organizations. That is, the patient primarily updates only the information associated with their electronic health record at the healthcare organization providing the interface.

The patient, therefore, cannot manage his or her healthcare from a single access point, but rather must use websites or other access points and corresponding accounts, including different usernames and passwords, for each individual healthcare provider organization. There is no central location that enables either a patient or a healthcare provider to update patient information that is synced across healthcare organizations in a network, or to provide consistent preference information, such as best way to contact the patient.

As a result, patients are often required to access multiple web sites to update their own patient data. The patients, moreover, are often required to repeatedly answer the same questions—to provide updates to personal data and information, provide insurance information, validate contact information, describe medical conditions, etc. The patients, further, may be required to repeatedly verify their identity through complicated verification processes, which may limit their use of online systems.

The disclosed system addresses these and other issues.

### SUMMARY OF THE DISCLOSURE

The disclosed system enables a patient or other authorized user to access and change health records stored in health information systems at different healthcare organizations from a single, secured central account or access point. The secured central account is configured to communicate to other electronic healthcare record systems via secure links and/or application programming interfaces (APIs). The system can receive information from a patient or other user at the central patient account and push this data into, for example, patient records maintained in EHR systems in a network in which each healthcare organization maintains separate patient health record databases.

The disclosed system comprises a distributed network of electronic health record systems which can operate the same health record system software, or which share information using shared standards. In addition to the health record systems, the system includes an additional, central node on the system which is accessible by patients and which can be used to establish a central account. Unlike typical nodes on a distributed network of electronic health record systems, the central node is not associated with a specific healthcare organization. The central node, however, is pre-authenticated to the other electronic health record systems, and can be identified by a unique code such as a health system identifier code that identifies other electronic health record systems in the network. The architecture of the system therefore differentiates from typical networks, which interconnect only systems that are associated with specific healthcare organizations. Because the central node is authenticated to the distributed network, however, the central node can be used by a patient to establish and access patient records at healthcare organizations throughout the network, thereby avoiding problems with maintaining updated data encountered in prior art systems.

To establish an account, the disclosed system can provide a user interface that enables a user to enter information to verify the identity of the patient. Once the user's identity is established, the user can establish a secure account protected, for example, with authentication data such as a username and password, biometric authentication, or other methods. Because the account is verified and authenticated to the patient, communications from the account are authorized to change demographic and contact information like the patient's name, address, or phone number, and the patient can directly authorize the submission of patient data to healthcare organizations through the account as a centralized access point. The user can further provide authorization to synchronize patient data among healthcare organizations, and/or identify the healthcare organizations maintaining records to be synchronized. The central patient account, therefore, enables updating patient records more easily, more quickly, more efficiently, and more thoroughly.

By providing direct access to each of the healthcare organizations that the patient visits, it is also possible to provide a central display for the patient that manages appointments, billing information, and other information from their medical records from a number of healthcare organizations through a single interface.

The user, further, can update, verify, or correct patient data like addresses, telephone or cell phone numbers, email addresses and other electronic contact information, preferred methods of contact, and insurance information from a single interface, and push this information to selected EHRs in the networked system, or all EHRs in the networked system. The user may also have access to review medical charges, insurance payments, and other data.

The disclosed system, moreover, enables a patient to select healthcare organizations to receive data, and to “push” data to those organizations to update information at any time. The patient can also selectively turn off transmission of information entirely. The proactive push of information sends the data immediately when it is updated, and only when it is updated, allowing each organization to maintain the most recent information even outside of a treatment event while also avoiding the challenges and inefficiencies associated with query frequency.

The disclosed system also allows a patient to select which types of data they would like to transmit from the central node to each healthcare organization. For example, in some cases, a patient may want to update contact information at a healthcare organization, but choose not to transmit clinical information, such as data related to medical procedures performed, to that same organization.

In situations where data cannot be entered by patients due to internal rules at a healthcare organization, patients can still update it in the central node and then data that can be transmitted can still be transferred, and an indication that some parts of the update could not be accepted can be provided to the patient. If a healthcare organization requires a review of patient entered data prior to entry into a database, the disclosed system can provide a notification to the healthcare organization to begin the process.

When healthcare organizations use specific internal data storage identifiers, the system can include mapping arrays or tables to translate data to and from the internal data storage identifiers. The system can maintain data using existing standards, such as Health Level 7 (HL7) or Systematized Nomenclature of Medicine (SNOMED).

In one aspect, a distributed network of electronic health record systems is disclosed. The network comprises a plurality of electronic health record systems connected in a network, each of the electronic health record systems adapted to communicate with other electronic health record systems in the network via secure communication links; and a central network node in communication with each of the plurality of electronic health record systems, the central network node including or in communication with a processor configured to: display a user interface to a patient, the user interface configured to receive data from the patient to authenticate an identity of the patient; establish a central patient account for the patient, the central patient account being pre-authorized to access each of the plurality of electronic health record systems; and to change patient data in an electronic health record associated with the authenticated patient requesting the change at each of the plurality of electronic health record systems.

Each of the plurality of electronic health record systems and the central node can be pre-authenticated to one another. The central node can be further configured to establish links from the central patient account to each of the electronic health record systems in the plurality of electronic health record systems that the patient corresponding to the central patient account has an account with.

The central node can comprise a server or a virtual server on the network, a memory component, and a communications system configured to communicate with the electronic health record systems in the network. The communications links can be provided via at least one of the internet, a wide area network, satellite communications, or cellular communications.

The central node can be further configured to develop an authentication confidence score for the patient based on at least one of patient demographic or identification data, number of organizations who have seen the patient, the patient's verification status at each organization, and the depth of clinical history associated with the patient's record across organizations.

The central node can be further configured to establish links between the central patient account and at least one of the plurality of electronic health record systems that maintains a patient record for the patient.

The central node can be further configured to retrieve rules for updating patient records at healthcare organizations corresponding to each of the plurality of electronic health record systems, and to transmit a message for the patient in the event that updates were not able to be immediately processed by a healthcare organization.

The central node can be further configured to send an identity of the patient and authenticate the patient to enable access to data at one or more of the plurality of health record systems.

The central node and at least one of the plurality of electronic health record systems can be in communication through a secure link, the secure link being encrypted using HTTPs. The secure link can be established using mutual transport layer security (TLS). The central node and each of the electronic health record systems can be nodes in the distributed network, and each node can present a client certificate to the other nodes and receives approval from the other nodes to establish the secure links.

Access from at least one of the plurality of electronic health record systems to the central node can be granted through an application programming interface (API).

Shared standards can be used to exchange data between the central node and the at least one of the plurality of electronic health record systems.

The central node can be configured to generate a request with a token corresponding to a set of demographics associated with the patient using the user interface and a health system identifier identifying the central node. The demographic data can be retrieved from memory associated with the central node or from a patient entering the demographic data directly into the user interface. The central node can be configured to store the token in memory, and to transmit a token identifier to at least one of the plurality of electronic health record systems with the health system identifier associated with the central node. The electronic health record system can further be configured to identify the central node from the health system identifier.

In another aspect, the disclosure provides a distributed network of electronic health record systems. The network comprises a plurality of electronic health record systems connected in a network, each of the electronic health record systems associated with a health system identifier identifying a healthcare organization, each of the electronic health record systems adapted to communicate with other electronic health record systems in the network via secure communication links established that use the health system identifiers to identify the electronic health record systems in the network; and a central network node in communication with each of the plurality of electronic health record systems. The central network node is independent of each of the plurality of healthcare organizations in the network, and is associated with a unique health system identifier identifying the central node as a member of the distributed network. The central network node includes a processor configured to: display a user interface configured to authenticate an identity of the patient to the central node; establish a central patient account for the patient; and establish a secure communication link with at least one of the plurality of electronic health record systems, wherein a patient at the user interface is authenticated to the electronic health record systems in the network, and wherein the patient is authorized to enter or change at least some of the patient data stored in the electronic health record systems in the network.

In yet another aspect, a method for updating patient demographic data across a distributed network of electronic health record systems is disclosed. The method comprises the steps of: associating each of a plurality of electronic health record systems with a unique healthcare system identifier identifying a healthcare organization corresponding to the electronic health record system; associating a unique healthcare system identifier with a central node in the network that is independent of each of the healthcare organizations in the distributed network, wherein the central node is configured to communicate with the electronic health record systems in the network as a peer; providing patient access to the central node, and receiving data from a patient to authenticate an identity of the patient; authenticating an identity of the patient; receiving updated patient data from the patient to update the patient's electronic health record; establishing secure communications links with the electronic health record systems in the network that are associated with the patient; and transmitting the updated patient data to each of the electronic health record systems in the network that are associated with the patient and storing the updated data in an electronic record corresponding to the patient.

These and other aspects of the disclosed system are described more fully below.

### BRIEF DESCRIPTION OF THE DRAWINGS

FIG. 1 is a block diagram of a prior art distributed network of electronic health record systems;

FIG. 1A is an illustration of an electronic health record system including a patient portal infrastructure corresponding to a healthcare organization;

FIG. 2 is a block diagram of the network of electronic health record systems of FIG. 1 illustrating the addition of a central patient account constructed in accordance with the current disclosure;

FIG. 3 is a block diagram of a network of electronic health record systems in which patient access is provided through a central node;

FIG. 4 is a flow chart illustrating an authentication process for authenticating a user through the central node;

FIG. 5 is a flow chart illustrating the process steps for bootstrapping patient data stored in the network to an account established by the patient using the central node.

FIG. 6 is a flow chart illustrating the process steps for completing the establishment of a patient record at a new health care organization.

### DETAILED DESCRIPTION

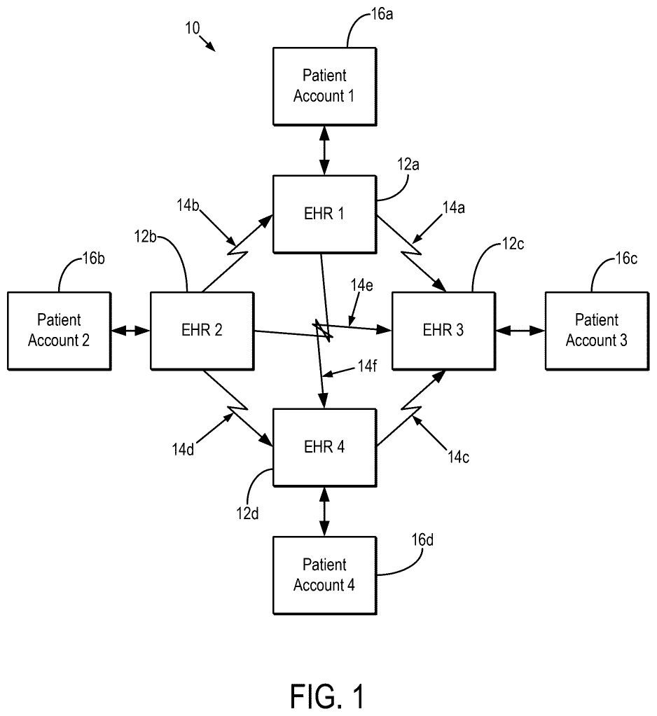

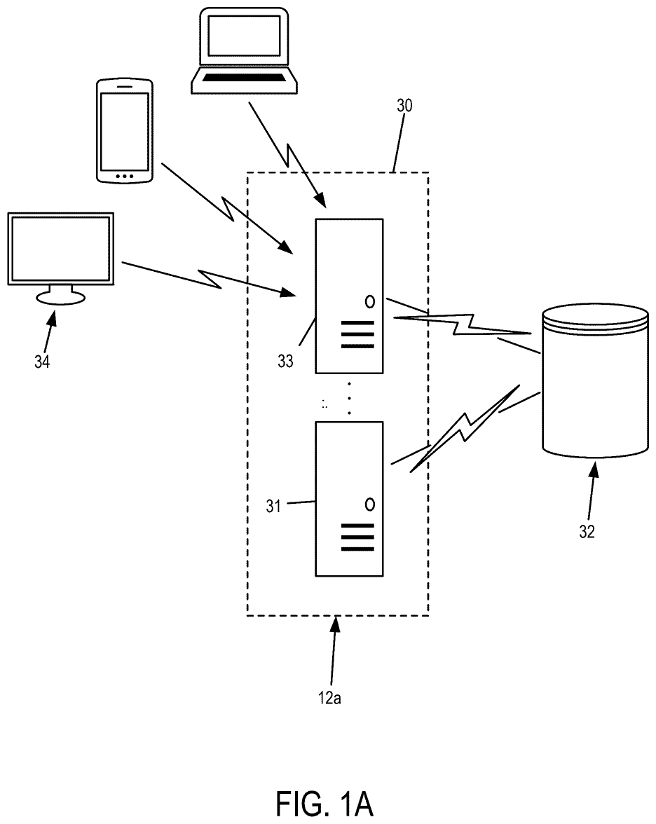

Referring now to FIG. 1, a block diagram of a prior art distributed network 10 of electronic healthcare systems is shown. As illustrated here, a plurality of healthcare organizations each operate a corresponding electronic health record system (EHR) 12a, 12b, 12c, 12d, storing patient records for patients that have been seen at the corresponding healthcare system. The healthcare organizations are each identified by a health system identifier, which is a unique code assigned to the organization for identification purposes. The health system identifier, in combination with a patient id (or code) from that system, creates a unique patient-organization combination. Each of the EHRs in the network is also authenticated to the other EHRs in the network using encryption algorithms, and digital certificates that are provided to participants. A central database or “phone book” provides contact information for participating healthcare systems, so that communications between the EHRs can be trusted by the other EHRs in the network. The EHRs 12a, 12b, 12c, 12d include implementations, deployments, or instances of software configured to maintain and access electronic health records which can be housed, for example, on a dedicated server, or accessed to provide a virtual machine through a software as a subscription model, by way of example. Although four EHRs are illustrated in FIG. 1, it will be apparent that the illustration is only an example, and any number of EHRs can be connected within a network.

Referring now also to FIG. 1A, a diagram of a typical healthcare information system housing EHRs is shown, here illustrated by way of example as EHR 12a. As illustrated here, each EHR may have server infrastructure 30 for storing and maintaining electronic health records in a local or remote memory storage device 32, and communications access for clinicians and other staff to interact with and update patient information either directly or through interconnected computing devices 34, which may include, as illustrated, interactive displays connecting directly to the server; or computers that include internal processors, memory components, and a user interface, which can be or include a touchscreen, keyboard, mouse, wi-fi connection and/or other interface components. Suitable computing devices include, but are not limited to, laptop and desktop computers, and personal computing devices such as tablets or smartphones.

Referring still to FIG. 1A, the server infrastructure 30 can include one or more servers, here illustrated as servers 31 and 33. In the illustrated embodiment, the server infrastructure 30 can manage connections to other applications, processes, or end users, and can include a server or servers 31 responsible for running a patient portal application that can be used by patients to access and update patient information. Patients can use their own computing devices 34 such as smartphones, laptops, or desktops to access server 31, and can view and interact with the EHR's patient portal. One or more additional server(s) can provide various functions, including communications and management functions. For example, server 33 can provide access to memory storage device 32, which as a non-limiting example may include a database, for clinical and administrative staff, or provide interoperability with other healthcare organizations through, for example, connections with EHRs 12b, 12c, and 12d. Server 33 can also provide access to Fast Healthcare Interoperability Resources (FHIR) resources which provide rules and specifications for exchanging electronic healthcare data; and can provide management of deployments and updates of EHR software. While two servers, 31 and 33 are illustrated, in practice any number of servers could be set up for these purposes within a specific EHR implementation

Each of the EHRs 12a, 12b, 12c, 12d in the network 10 act as nodes, e.g. devices or virtual devices creating, receiving, or transmitting data on the network 10. The EHRs 12a, 12b, 12c, and 12d can include corresponding processing devices, and either include or are in communication with memory storage devices 32, which include data structures for storing patient health data and other types of related data including but not limited to scheduling and insurance data corresponding to the patients. The EHRs 12a, 12b, 12c, 12d can further include communications devices, and are configured to communicate with other EHRs in the system through wired or wireless communications systems via secure links 14a, 14b, 14c, 14d, 14e, 14f. These links can, for example, be provided through the internet, across wide area networks, through cellular or satellite communications systems, or in other ways for providing electronic communications, which will be apparent to those of skill in the art. These connections can be encrypted, for example, using HTTPs. Trust between parties can be established using mutual transport layer security (TLS) where the initiating organization must present a client certificate and the receiving system must have their own certificate. Each side must review and approve the other's certificate. Each of the EHRs 12a, 12b, 12c, 12d includes or has access to a database that stores patient data records for the patients of the corresponding healthcare organization, which can be in a memory storage device 32 associated with the corresponding EHR, or in a remote location such as cloud storage. When authorized by a patient at the point of care, or through other forms of authorization, each of the EHRs 12a, 12b, 12c, 12d can use the secure links 14a-14f to query other EHRs in the network for treatment data corresponding to the patient that authorized the access. In some cases, the patient records in each EHR corresponding to the patient can be updated with a list of all the healthcare providers that are known to have seen the patient, along with corresponding patient identifiers. Although this process provides a significant improvement in maintaining the currency of patient records, the records are typically only updated when the patient seeks treatment at the healthcare organization. The healthcare organizations often cannot query to update data unless they are currently treating the patient, and stored patient data will therefore not remain current if the patient doesn't regularly seek treatment with all the healthcare organizations storing data for the patient. Further, although the healthcare organization that is currently treating the patient can update its records, the records held by the other healthcare organizations that have treated the patient are not typically updated in this process.

Referring still to FIG. 1 and to FIG. 1A, each of the EHRs 12a-12d can also present a user interface to a connected computing device 34 that is configured to receive information to establish a patient account. For example, the EHRs 12a-12d may operate a web site which presents a web page or other user interface to a patient and allows the patient to establish and access a local account 16a, 16b, 16c, 16d that is housed at the corresponding EHR. As part of the account establishment process, the patient typically will establish a username and a password to enable later access to the system.

The healthcare organization associated with the corresponding EHR can store rules establishing the types of information that can be accessed and changed via the patient user interface, and data access by the user is therefore restricted by these rules. Typically, patients can access and change personal data, including, for example, address, telephone numbers, and insurance information, from the patient account corresponding to the EHR. Patients may also be able to access schedules and patient data, including the results of tests. In some applications, patients may also be able to set preferences, such as their preferred method of receiving communications. Although, as described above, patient data can be updated from connected EHRs when the patient seeks treatment, this data will not be updated unless the patient returns for treatment. Therefore, if a patient changes an address at patient account 16a, the address is changed in EHR 12a. Unless the patient is also subsequently seen for treatment at healthcare organizations corresponding to EHRs 12b-12d, these healthcare organizations will not have a current, updated address for the patient unless they log into their patient accounts 16b-16d to change it in each system.

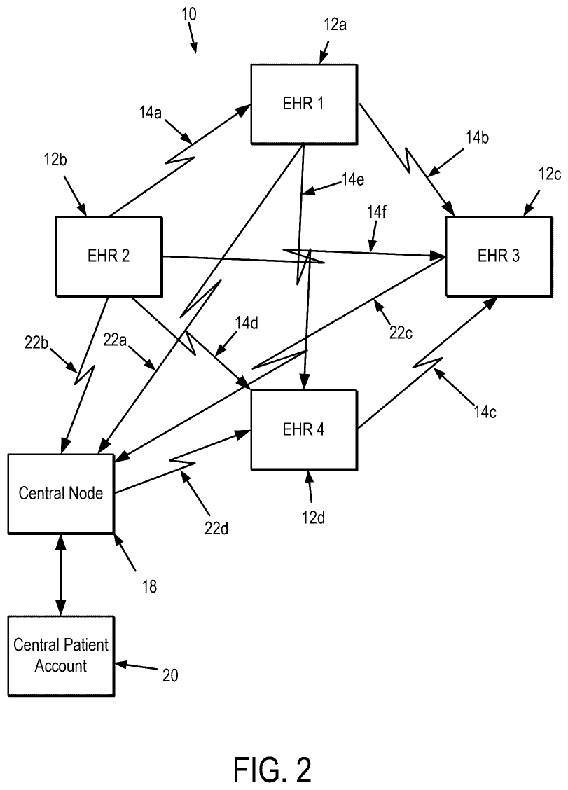

Referring now to FIG. 2, a network of electronic healthcare systems constructed in accordance with the disclosure is shown. The network includes EHRs 12a, 12b, 12c, 12d, as described above. The EHRs 12a, 12b, 12c, 12d are, again, nodes on the network, and are configured to communicate with one another through secure links 14a, 14b, 14c, 14d, 14e, 14f. Here, in addition to EHRs 12a, 12b, 12c, 12d, an additional central node 18 is provided on the network 10. The central node 18 includes or provides access to processing, communication, and memory storage devices, and is in communication with each of the EHRs 12a, 12b, 12c, 12d via communication links 22a, 22b, 22c, 22d. As described above, these communication links can be encrypted, and trust between the central node 18 and other parties can be established using mutual transport layer security (TLS). Each of the EHRs 12a, 12b, 12c, 12d and the central node 18 are pre-authenticated to the other nodes in the network, so that communications between each of the nodes can be trusted by other nodes in the system. Although the central node is not associated with a healthcare organization, the central node can also be identified with a health system identifier, to enable identification in communications with the other nodes in the system. The central node 18 is programmed to provide a central user interface configured to receive information from a patient to establish a central patient account, either directly or through an interconnected device.

For example, the central node 18 may present a web page configured to verify the identity of a patient for establishing a central patient account 20. To verify the identity of the patient, the central node 18 and EHRs 12a, 12b, 12c, 12d can include a shared identity verification algorithm that uses data associated with the patient as a normal part of receiving healthcare to verify the identity of the patient, thereby avoiding additional steps for users. The algorithm can evaluate a number of factors including but not limited to the number of healthcare organizations in the network who have seen the patient, the patient's verification status at each organization, and the depth of clinical history associated with the patient's record across organizations, and produce a confidence score for how likely a user accessing the central node 18 is who they say they are based on the available data. Organizations operating EHRs 12a, 12b, 12c, 12d on the network can access the algorithm and corresponding confidence factors to determine if the data meets their internal identity verification standards, and accept or reject the shared verification of the patient accordingly.

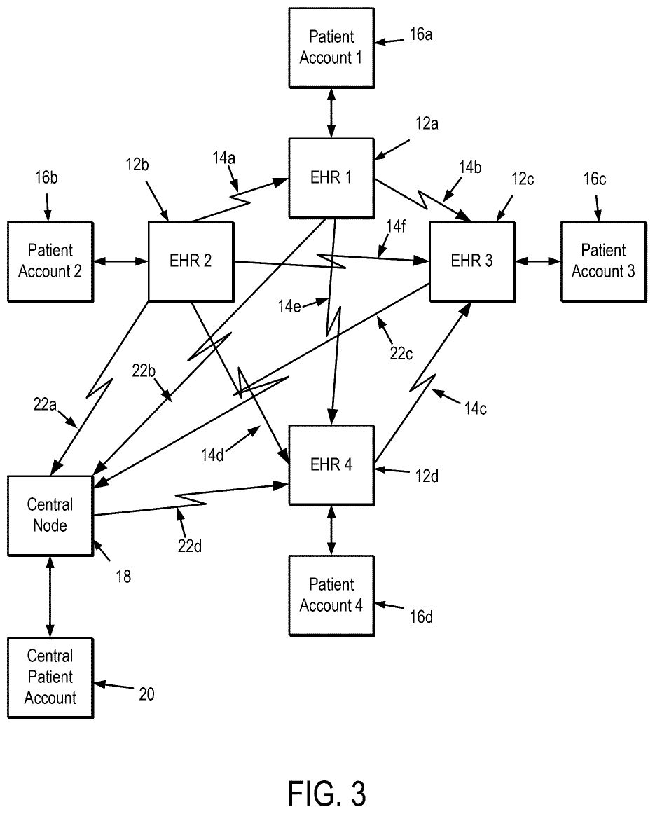

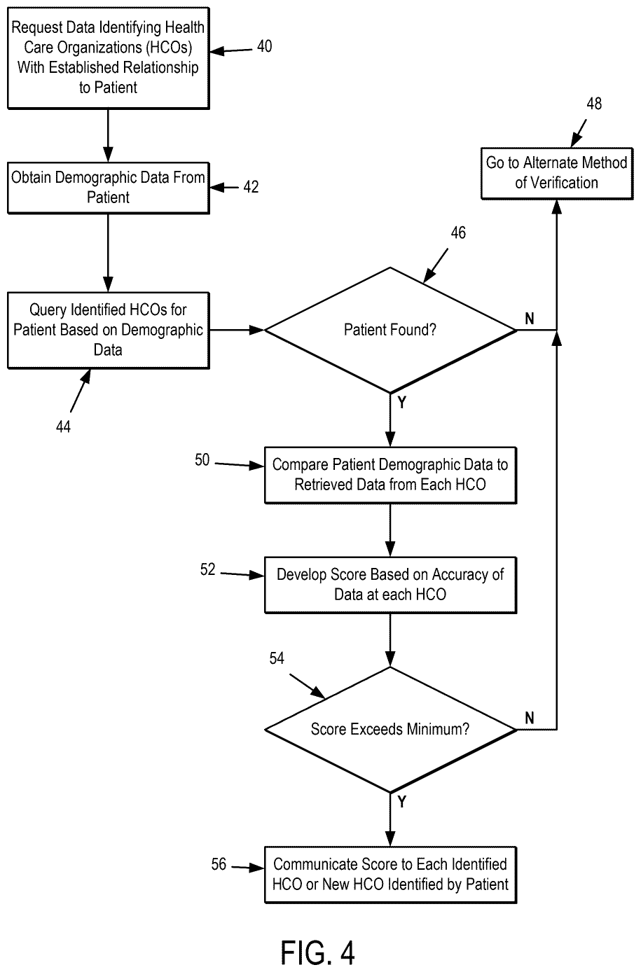

Referring now to FIGS. 3 and 4, by way of example, when a user accesses the central node 18, the central node 18 can query the patient to identify the healthcare organizations that patient has seen in the past, and therefore has a pre-existing patient relationship with (step 40). The central node 18 can also query the user for demographic data and/or personal data, such as a name, address, social security number, driver's license number, email address(es), and/or telephone number(s) (step 42). Prior to granting the user access to the information from each healthcare organization the algorithm can then query the identified healthcare organizations to identify patient records (step 44), and determine whether the patient can be found in records corresponding to the identified healthcare organizations (step 46). If the information supplied by the patient is sufficient to identify patient records, the central node 18 can retrieve both personal data that corresponds to the data entered by the patient and additional data stored in the identified patient records. (step 50) The central node 18 can then develop a confidence score about the patient's identity based on content of the data, specifically relating to the degree of certainty that the person accessing the online service is the same person as identified by the data (step 52). As described above, the number of healthcare organizations in the network who have seen the patient, the patient's verification status at each organization, and the depth of clinical history associated with the patient's record across organizations may also be considered in calculating the confidence score. The central node 18 can then evaluate whether the confidence score exceeds a predetermined minimum value, which can be a value established for every healthcare organization in the system, or which can be established individually by each healthcare organization.

Alternative verification processes can also be provided to the patient (step 48), for example, including, but not limited to, if the patient was not sufficiently identified in step 46, or the confidence score did not exceed the minimum in step 54. The alternative verification process can include any process to determine or increase the identity score, including but not limited to authenticating into an account that has already been verified to be held by the patient, providing additional identifying information such as government issued identification, or completing an identity verification process offered by organizations specializing in this. The confidence score and a summary of the data associated with the patient in each identified EHR can then be communicated to the EHRs 12a, 12b, 12c, or 12d identified by the patient (step 56). The individual EHRs can each determine whether the confidence score meets their threshold for accepting the identity of the patient. In some cases, such as when the verification level is below a specific organization's standards, a communication, such as an email or text message, may be generated and provided to an administrator at the healthcare organization to review the request.

As described above, in some cases an alternative, or additional, method of verification may be required. For example, the patient may be asked to provide additional forms of identifiers, including verifiable state issued documents, digital state IDs, proof of phone number ownership, proof of address control, or acceptable knowledge based verification such as social security numbers, date of birth, and/or a list of addresses which can be compared against databases to verify the identity of the patient. Also as described above, in some applications, patients may be identified based on querying EHRs 12a-12d to determine if the patient has already been positively identified at one or more of the healthcare organizations. Alternatively, the patient may be identified by verifying that the patient can authenticate themselves to any one or more of the patient accounts 16a-16d. In still other applications, patients may be positively identified by a third party authentication service. Suitable services are provided by Prove Identity, Inc., New York, NY; ID.me, of McLean, VA, and Clear Secure Inc., New York, NY. These services can be used, for example, when the patient's identity cannot be verified by the information provided.

When a central patient account 20 is established at the central node 18 and the patient's identity verified to a confidence level acceptable by each of the linked healthcare organizations, the central account 20 can therefore be authenticated and pre-authorized to access data corresponding to the patient at each of the EHRs 12a, 12b, 12c, and 12d, and can access other functions such as scheduling through communication links 22a, 22b, 22c, 22d from the central node 18. Typically, when the account 20 is established, the patient will be prompted to establish criteria for authenticating the patient, such as a username and password for accessing a central patient account 20.

When the patient is properly verified a link can also be established from the central account 20 to the accounts 16a-16d at each other organization. The central account 20 can then be used as an “identity provider.” When a patient who has established a central account 20 goes to the healthcare organization operating EHR 12d, for example, the patient can log in using the central account 20, with EHR 12d querying the central node 18 to authenticate the patient. The central node 18 can request authentication data, such as a username and password, and if the authentication is successful, the central node 18 replies to the query indicating that the patient has been successfully authenticated. The central username and password, therefore, can identify the patient across the network. Although the healthcare providers may choose to maintain individual patient accounts 16a, 16b, 16c, 16d as well as a central patient identifier, as illustrated in FIG. 2, the functions provided by the individual patient accounts 16a, 16b, 16c, 16d will also be available using only the central patient account 20 as illustrated in FIG. 3. That is, some actions could be taken by the patient from their central account 20 directly. If a patient chooses to go to the website of any given healthcare provider 12a-12d, they can also use their central account 20 to authenticate themselves and then access the same resources that are accessible through the local account 16a-16d corresponding to the EHR.

When accessing the EHRs on network 10 through the central node 18, the patient has been pre-authenticated by the system, and can make changes across each of the EHRs 12a, 12b, 12c, and 12d. The patient, therefore, can “push” data into their own patient records, enabling the patient to change personal data such as addresses, electronic addresses, telephone and cell phone numbers, and insurance information from a single location. In some applications, the healthcare systems associated with each EHR may have their own rules associated with reviewing and approving a change in patient data. The central node 18 can access and respect the rules established by each EHR and may, in some applications, maintain sets of rules for each healthcare organization, and prevent immediate entry of the change until the rules of the organization are met. The central node 18 may, instead of changing the information directly, forward a notification to the healthcare system that a change has been requested by the patient, thereby allowing the healthcare system to request reviewing and approving information from the patient consistent with its own rules for receiving external data. Alternatively, the EHRs 12a-12d may each maintain their own rules, and evaluate incoming data to determine whether the rules have been met.

To ensure portability of patient records, and to ensure that the patient records at each of the EHRs 12a, 12b, 12c, and 12d are current, the patient can authorize synchronization of their patient data at each of the healthcare organizations from the central patient node 18, and “push” updated information to other linked organizations in near real-time. Here, for example, the patient can provide a list of healthcare organizations that have records for the patient, and authorize updates of their data from the central node to each organization, including updates and clarifications to patient demographics such as name, address, and/or personal data. After the account is established, the central node 18 can automatically maintain a list of healthcare organizations for the patient who holds the account. In some applications, the patient can also choose to store select patient data locally in memory at the central node 18. Local patient data may be used, for example, to allow patients to track data from personal devices, including wearable devices, in their central account.

Once the patient has established a central account 20 and has been verified and authenticated, the central account 20 can also be used by the patient to establish care at a new healthcare organization. Assume, for example, that the patient has established a central account, and has been seen by the healthcare organizations corresponding to EHRs 12a, 12b, and 12c, and now seeks to establish care at the healthcare organization corresponding to EHR 12d.

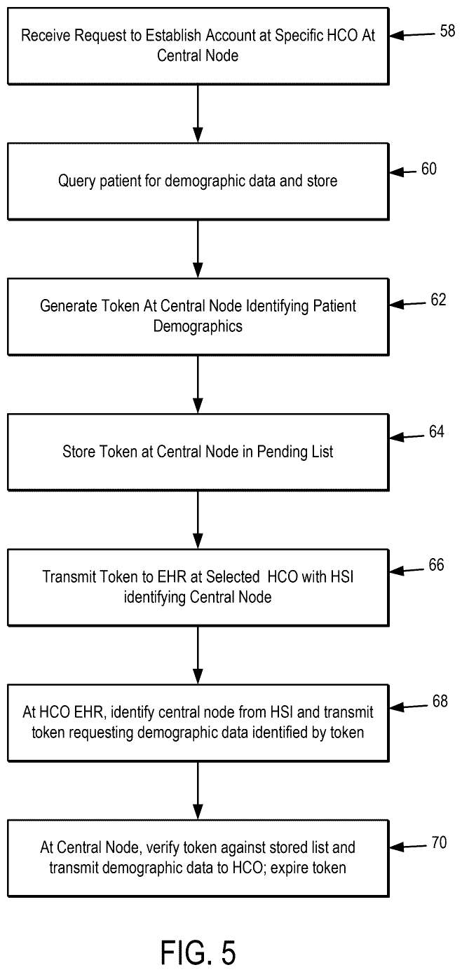

Referring now to FIG. 5, when the user seeks to establish care (step 58), the central node 18 generates a request with a token corresponding to a set of demographics and/or personal data associated with the patient establishing the account and an identifier corresponding to a health system (health system identifier). The demographic data and/or personal data can be retrieved from central node 18 or can be entered by a patient directly if the patient does not have any established accounts (step 60). The central node 18 stores the token in memory, and then transmits the token identifier to EHR 12d with the health system identifier associated with the central node or another unique identifier identifying the central node to other systems in the network (step 66). The central node 18 can trust EHR 12d because EHR 12d had to be authenticated to join the network 10, as described above. Other EHRs 12a-12c in the network can trust the central node 18 because the central node has also been authenticated to the system.

When the EHR 12d receives the request from central node 18, it identifies central node 18 from a health system identifier attached to the request and transmits the token as part of a request for demographic data and/or personal data corresponding to the token (step 68). Other EHRs 12a-12c in the network can trust the central node 18 because the central node has also been authenticated to the system.

The central node 18 receives the token from EHR 12d and compares it against the pending tokens it has generated. If there is a match, central node 18 responds with the set of known demographic data and/or personal data and expires the token (step 70).

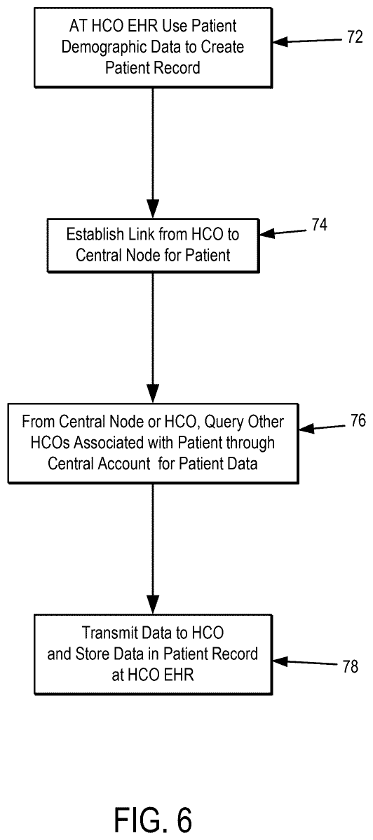

Referring now to FIG. 6, the EHR 12d receives the demographic data and/or personal data from the central node 18. It can create the patient record at the EHR 12d and can initiate a link to central node 18 by querying central node 18 on behalf of the user (step 72). Alternatively, if there are existing records in EHR 12d with similar demographic data and/or personal data, the system will attempt to connect to those existing records.

Once the link to central node 18 is established (step 74), central node 18 can propagate links to additional healthcare organizations to also attempt to link them to the EHR 12d, the new healthcare organization for the patient. Using these new links, the linked healthcare organizations can transmit patient data to the new healthcare organization's EHR 12d or central node 18 can query the EHRs corresponding to the healthcare organizations that the patient has an established relationship with to quickly populate the patient's chart for the new organization, and transmit the data to EHR 12d for storage (step 76).

On a user display screen used to access EHR 12d using central account 20, the process of populating a patient chart at the new EHR 12d may appear like a new web page load. As the remote EHRs 12a-12c link to EHR 12d, EHR 12d creates the patient record and responds with an appropriate page from which the user can create a patient account for the healthcare organization corresponding to EHR 12d, while the data transfer from the patient's pre-existing healthcare organizations occurs.

The patient, therefore, can authorize the new healthcare organization to access to their patient information and records through the central patient account 20. The new healthcare organization can access demographic data and/or personal data, such as patient name, address, and contact data, insurance information, a list of other healthcare organizations where the user is a patient through the patient central account 20, and/or directly access patient medical records. The disclosed system, therefore, improves portability and accuracy of patient records because the patient can be pre-identified and authenticated for healthcare organizations accessing the system to the level of certainty ascertained from the identity verification algorithm, and a current list of healthcare organizations that the patient uses or has used is readily available, so a healthcare organization seeking to update a patient record can easily identify other organizations to query for updated data. This allows the patient to establish a comprehensive and up to date record at healthcare organization at any time, even if the records of a different healthcare organization or central location do not include a comprehensive and up to date patient record. A central account 20 can be particularly useful to enable healthcare organizations to access patient records where a patient has moved or is traveling, and when the healthcare organizations they have used in the past are not in the same geographic location, or easily identified through a query. The patient, again, does not have to repetitively enter pre-existing data.

The central patient account may also be used to access services at each of the healthcare organizations associated with EHRs 12a, 12b, 12c, and 12d. The central patient account may, for example, be used to identify new providers or specialists across healthcare organizations. Patients may also use a central account 20 to enroll or participate in remote patient monitoring or care at home programs. Other functions that can be performed from a central account 20 can include accessing scheduling at each of the corresponding EHRs and providing a schedule or calendar of all services. In addition, the patient may be provided the option of scheduling medical appointments, lab services, rehabilitation or clinical therapies through the central patient account 20.

The central patient account may also have access to medical invoices or expenditures at each healthcare organization. Here, the central account 20 may be used by the patient to generate or view estimates for services, manage payment plans, apply for financial assistance. The system may also track insurance information, such as the current status of deductibles, by enabling the patient to access total current expenditures for all providers.

In addition, where a user is a guardian or holds a medical power of attorney for other patients, the central node 18 can determine that the user has established proxy access for another patient, and access to records for that patient can be provided to user. Alternatively, the user can elect to add access for dependents to the account associated with the user. Here, for example, the user could select an option to add a patient to an account, provide data to identify the patient, and provide information to establish that the patient is a dependent of the user. The user would then be granted proxy access to the dependent's account. Access for additional accounts could be provided though a link, access to a tab on a display screen, or by providing a separate window, by way of example. The user may also be provided the option to produce a combined calendar of appointments for all the patients in the linked accounts, combined expenditure lists, etc.

Where healthcare organizations maintain separate patient account access, as illustrated in FIG. 2, links to these individual organization web sites may be provided on a user interface provided when accessing a central account 20 through the central node 18.

Although access to healthcare organizations would generally be restricted to organizations operating instances of the same EHR software, access to patient data, scheduling applications and other information in other EHR systems may be provided to the central patient account through access granted via application programming interfaces (APIs). In these cases, shared standards can be used to exchange data among healthcare organizations.

Referring again to FIG. 2, when individual patient accounts 16a, 16b, 16c, and 16d, and a central account 18 are used, the central username and password established for a user through the central node 18 may also be used to access the corresponding healthcare system EHR from the patient accounts 16a, 16b, 16c, and 16d established by the healthcare system. In this case, an independent web site for the healthcare organization can be maintained while also simplifying access to the individual healthcare systems for the patient.

As described above, memory associated with the central node 18 can be used to store a database, tables, or mapping arrays to translate internal data storage identifiers used by different healthcare organizations. A central database of connected organizations and corresponding health system identifiers can also be stored at the central node. Additionally, the central database could be used to retrieve and store additional patient-generated clinical data, centrally managed patient services, up to date payer and plan information, or information from across the network such as a list of clinical trials available across healthcare organizations.

Although the system has been described above for use by a patient, a central node that is authorized to push data onto the electronic health record systems operating on a network can also be used in other applications. For example, pre-authorized access to updated insurance guidelines, billing codes, or other systems can also be provided using similar methods.

1. A distributed network of electronic health record systems, the network comprising:
a plurality of electronic health record systems connected in a network, each of the electronic health record systems adapted to communicate with other electronic health record systems in the network via secure communication links;
a central network node in communication with each of the plurality of electronic health record systems, the central network node including or in communication with a processor configured to:
display a user interface to a patient, the user interface configured to receive data from the patient to authenticate an identity of the patient;
establish a central patient account for the patient, the central patient account being pre-authorized to access each of the plurality of electronic health record systems, and to change patient data in an electronic health record associated with the authenticated patient requesting the change at each of the plurality of electronic health record systems.

2. The distributed network of claim 1, wherein each of the plurality of electronic health record systems and the central network node are pre-authenticated to one another.

3. The distributed network of claim 1, wherein the central network node is further configured to establish links from the central patient account to each of the electronic health record systems in the plurality of electronic health record systems where the patient corresponding to the central patient account has an account.

4. The distributed network of claim 1, wherein the central network node comprises a server or a virtual server on the network, a memory component, and a communications system configured to communicate with the electronic health record systems in the network.

5. The distributed network of claim 1, wherein the communications links are provided via at least one of the internet, a wide area network, satellite communications, or cellular communications.

6. The distributed network of claim 1, wherein the central network node is further configured to develop an authentication confidence score for the patient based on at least one of patient demographic data or personal data, number of organizations who have seen the patient, the patient's verification status at each organization, and the depth of clinical history associated with the patient's record across organizations.

7. The distributed network of claim 6, wherein the central network node is further configured to establish links between the central patient account and at least one of the plurality of electronic health record systems that maintains a patient record for the patient.

8. The distributed network of claim 1, wherein the central network node is further configured to retrieve rules for updating patient records at healthcare organizations corresponding to each of the plurality of electronic health record systems, and to transmit a message for the patient in the event that updates were not able to be immediately processed by a healthcare organization.

9. The distributed network of claim 1, wherein the central network node is further configured to send an identity of the patient and authenticate the patient to enable access to data at at least one of the plurality of health record systems.

10. The distributed network of claim 1, wherein the central network node and at least one of the plurality of electronic health record systems are in communication through a secure link, the secure link being encrypted using HTTPs.

11. The distributed network of claim 10, wherein the secure link is established using mutual transport layer security (TLS).

12. The distributed network of claim 10, wherein the central node and each of the electronic health record systems are nodes in the distributed network, and each node presents a client certificate to the other nodes and receives approval from the other nodes to establish the secure links.

13. The distributed network of claim 1, wherein access from at least one of the plurality of electronic health record systems to the central node is granted through an application programming interface (API).

14. The distributed network of claim 13, wherein shared standards are used to exchange data between the central node and the at least one of the plurality of electronic health record systems.

15. The distributed network of claim 1, wherein when the patient accesses the user interface to establish an account, the central node is configured to generate a request with a token corresponding to a set of demographic data or personal data associated with the patient and a health system identifier identifying the central node.

16. The distributed network of claim 15, wherein demographic data or personal data is retrieved from memory associated with the central node or from a patient entering the demographic data or personal data directly into the user interface.

17. The distributed network of claim 15, wherein the central node is configured to store the token in memory, and to transmits a token identifier to at least one of the plurality of electronic health record systems with the health system identifier associated with the central node.

18. The distributed network of claim 17, wherein the at least one of the plurality of electronic health record systems is configured to identify the central node from the health system identifier.

19. The distributed network of claim 1, wherein the central network node is configured to enable patients to access billing, scheduling, and remote patient monitoring from a plurality of healthcare organizations in the network from a central account on the central network node, without accessing the corresponding healthcare organization's local portal.

20. A distributed network of electronic health record systems, the network comprising:
a plurality of electronic health record systems connected in a network, each of the electronic health record systems associated with a health system identifier identifying a healthcare organization, each of the electronic health record systems adapted to communicate with other electronic health record systems in the network via secure communication links established that use the health system identifiers to identify the electronic health record systems in the network;
a central network node in communication with each of the plurality of electronic health record systems, the central network node being independent of each of the plurality of healthcare organizations in the network, the central network node being associated with a unique health system identifier identifying the central node as a member of the distributed network, the central network node including or in communication with a processor configured to:
display a user interface to a patient, the user interface configured to receive data from the patient to authenticate an identity of the patient to the central node;
establish a central patient account for the patient; and
establish a secure communication link with at least one of the plurality of electronic health record systems, wherein a patient at the user interface is authenticated to the electronic health record systems in the network, and wherein the patient is authorized to enter or change at least some of the patient data stored in the electronic health record systems in the network.

21. A method for updating patient demographic data or personal data across a distributed network of electronic health record systems, the method comprising:
associating each of a plurality of electronic health record systems with a unique healthcare system identifier identifying a healthcare organization corresponding to the electronic healthcare record system;
associating a unique healthcare system identifier with a central node in the network that is independent of each of the healthcare organizations in the distributed network, wherein the central node is configured to communicate with the electronic health record systems in the network as a peer;
providing patient access to the central node, and receiving data from a patient to authenticate an identity of the patient;
authenticating an identity of the patient;
receiving updated patient data from the patient to update the patient's electronic healthcare record;
establishing secure communications links with the electronic health record systems in the network that are associated with the patient; and
transmitting the updated patient data to each of the electronic health record systems in the network that are associated with the patient and storing the updated data in an electronic record corresponding to the patient.

22. A method for improving portability of patient data in a distributed network of electronic health record systems, the method comprising:
establishing a central patient account authenticating a patient to the electronic health record systems in the network;
establishing authenticated communication links between the central patient account and at least one electronic health record system in the distributed network;
establishing a new patient record at the at least one electronic health record system;
using authenticated communication links from the central patient account to retrieve records corresponding to the authenticated patient from electronic health record systems in the distributed network, and storing the records in the new patient record, wherein the new patient record includes current data from each of the electronic health record systems in the distributed network.
